(19) United States
(12) Patent Application Publication
(10) Pub. No.: US 2005/0013845 A1
(43) Pub. Date: Jan. 20, 2005

(54) Adhesive bioerodible ocular drug delivery system

(76) Inventors: Stephen L. Warren, Fort Collins, CO (US); David W. Osborne, Santa Rosa, CA (US); Richard Holl, Fort Collins, CO (US)

Correspondence Address: Schwegman, Lundberg, Woessner & Kluth, P.A., P.O. Box 2938, Minneapolis, MN, 55402, US

(21) Appl. No.: 10/706,603

(22) Filed: Nov. 12, 2003

Related U.S. Application Data
(60) Provisional application No. 60/425,508, filed on Nov. 12, 2002.

(51) Int. Cl. A61F 6/06

(52) U.S. Cl. 424/430

(57) ABSTRACT

The present invention provides bioerodible, water-soluble pharmaceutical carriers for ocular (e.g., transconjunctival or transcorneal) delivery of pharmaceuticals for either systemic or local therapy.

### RELATED APPLICATION

This application claims priority from U.S. Provisional Application No. 60/425,508; filed on Nov. 12, 2002; which is incorporated herein by reference.

### FIELD OF THE INVENTION

The present invention relates generally to bioerodible, water-soluble pharmaceutical carriers for ocular (e.g., transconjunctival or transcorneal) delivery of pharmaceuticals for either systemic or local therapy.

### BACKGROUND OF THE INVENTION

A number of mucoadhesive devices are available for the delivery of pharmaceuticals locally or systemically through a mucus membrane or within a mucosally lined body cavity. Many of these devices are in the form of a film or patch that conveniently fit within a cavity (e.g., mouth) and adhere to a mucus membrane. They are often designed to be pressure sensitive, and they adhere immediately upon application to a membrane.

The BEMA™ (Bioerodible Muco-Adhesive Film) Drug Delivery System is a bioerodible film for fast-acting local or systemic delivery of pharmaceuticals. The BEMA™ technology provides a mucoadhesive and bioerodible disc for application to a mucosal surface and is used for transmucosal delivery of pharmaceuticals over variable lengths of time, e.g., delivery occurring for minutes or hours. The BEMA technology is disclosed, e.g., in Tapolsky, et al. (U.S. Pat. No. 5,800,832) and Tapolsky, et al. (U.S. Pat. No. 6,159,498).

The treatment of the eye for disease and/or wounds requires that the particular pharmaceutical be maintained at the site of treatment for an effective period of time. Given the tendency of natural bodily fluids such as tears to rapidly wash away topically applied pharmaceutical components, local ocular therapy or use of the conjunctiva as a route for systemic administration has been problematic.

The use of ocular inserts for the delivery of drugs locally has been described for over 30 years (see, e.g., Ness, U.S. Pat. No. 3,416,530 and Cheng, U.S. Pat. No. 4,053,580). These original inserts included materials that were not soluble or bioerodible in tear fluids.

Other disclosures describe ocular delivery inserts that dispense drugs over a period of time and eventually are completely eroded, but none of these references have suitable bioadhesive capability. See, e.g., Whitaker, et al. (U.S. Pat. No. 3,963,025); Miyata, et al. (U.S. Pat. No. 4,164,559); Cohen, et al. (U.S. Pat. No. 4,179,497); Heller, et al. (U.S. Pat. Nos. 4,346,709 and 4,249,431); Darougar, et al. (U.S. Pat. No. 6,264,971); Wong, et al. (U.S. Pat. No. 6,331,313) and Masters (U.S. Pat. No. 6,342,250).

Flowable solutions of bioadhesive polymer mixtures have also been described to increase the residence time of eyedrops (Bowman et al., U.S. Pat. No. 6,372,245 and Chiou, U.S. Pat. No. 5,283,236). These solutions, however, do not maintain intimate contact with the conjunctiva to achieve rapid onset of therapeutic effects.

The eye is an anatomically complex organ that offers unique challenges and advantages for both the local and systemic delivery of pharmaceuticals. The surface epithelial tissues of the eye, the conjunctiva or cornea, are wet tissues constantly bathed with tears. This usually steady flow of moisture drains into the nasal lacrimal ducts at the medial canthus.

The eye's first response to a foreign object is increased tearing, which either washes the foreign matter out of the eye, or for pharmaceuticals in eye drops, washes the drug into the sinuses. The inner surface of the eyelid, or palpebral conjunctiva, is a moist, highly vascularized tissue. While the majority of pharmaceuticals in an eye drop drains from the sinuses into the back of the throat, some of the pharmaceutical will be taken into the vasculature and become systemic and some will penetrate through the bulbar conjunctiva to the anterior chamber of-the eye.

While transport into the systemic circulation is rapid, the efficiency of delivery from eye drops is low, and there is always potential for toxicity because topically applied drugs can readily gain access to the anterior segment of the eye.

Accordingly, what is needed is a bioerodible, water-soluble pharmaceutical carrier for ocular (e.g., transconjunctival or transcorneal) delivery of pharmaceuticals for either systemic or local therapy, over variable lengths of time, e.g., delivery occurring for minutes or hours. The carrier would preferably be in the form of a film or patch that would conveniently fit on an ocular surface. The carrier would preferably be pressure sensitive and would have suitable bioadhesive capability, such that it would adhere immediately upon application to an ocular surface. The carrier would maintain intimate contact with the conjunctiva, to achieve rapid onset of therapeutic effects. Additionally, the particular pharmaceutical would be maintained at the site of treatment for an effective period of time. Preferably, natural bodily fluids such as tears would not rapidly wash away topically applied pharmaceutical components, such that local ocular therapy or use of the conjunctiva as a route for systemic administration would not be problematic. The carrier would preferably be soluble or bioerodible in tear fluids.

### SUMMARY OF THE INVENTION

The present invention provides a bioerodible, water-soluble pharmaceutical carrier for ocular (e.g., transconjunctival or transcbrneal) delivery of pharmaceuticals for either systemic or local therapy, over variable lengths of time, e.g., delivery occurring for minutes or hours. The carrier is in the form of a film or patch that conveniently fits on an ocular surface. The carrier is pressure sensitive and has suitable bioadhesive capability, such that it adheres immediately upon application to an ocular surface. The carrier maintains intimate contact with the ocular surface (e.g., conjunctiva), to achieve rapid onset of therapeutic effects. Additionally, the particular pharmaceutical is maintained at the site of treatment for an effective period of time. Natural bodily fluids such as tears do not rapidly wash away topically applied pharmaceutical components, such that local ocular therapy or use of the ocular surface (e.g., conjunctiva) as a route for systemic administration is problematic. Additionally, the carrier is soluble and bioerodible in tear fluids.

The use of the bioerodable, water-soluble pharmaceutical carrier of the present invention has advantages compared to current mucosal drug delivery systems. Placing the drug loaded adhesive side of the bioreodible carrier of the present invention in contact with the bulbar conjunctiva or the corneal epithelium provides improved delivery to ocular tissues in the anterior and/or posterior segments of the eye. Placing the drug loaded adhesive side of the bioerodible carrier of the present invention in contact with the palpebral conjunctiva (the wet inner surfaces of eyelids), either superior or preferably the inferior palpebral conjunctiva, provides improved delivery to the systemic circulation. Administration to the inner surface of the eyelid is useful for localized delivery. For either localized or systemic drug delivery, the transocular (e.g., transconjunctival or transcorneal) route of administration provides for faster onset of drugs compared to other mucosal routes of drug administration.

By adhering to the ocular (e.g., conjunctival or corneal) surface, the carrier of the present invention creates intimate contact and excludes the tears from the area of contact. Bioadhesion also restricts the site of drug entry to the surface area covered by the carrier. This minimizes the amount of pharmaceutical washed away from the application site.

By placing a pharmaceutical in an adhesive, bioerodible drug delivery system, loss of the pharmaceuticals into the sinuses can be minimized while delivery via the palpebral conjunctiva can be maximized. Likewise, pharmaceuticals loaded into the adhesive layer of the bioerodible drug delivery system of the present invention, that is adherent to the bulbar conjunctiva, will maximize delivery into the eye.

The ability of the drug delivery system of the present invention to adhere to the conjunctiva is important to assure that a minimal amount of the applied pharmaceutical drains into the sinuses, which results in highly variable systemic and localized pharmaceutical delivery. The mechanics of adhesion itself may also promote drug delivery at the site of application. Relatively subtle effects of the adhesive on the epithelial barrier may promote the transport of pharmaceuticals.

The adhesive bioerodible drug delivery system of the present invention can be placed on either the inferior palpebral conjunctiva, also known as the tarsal conjunctiva, or the superior palpebral (tarsal) conjunctiva for systemic delivery of the pharmaceuticals. Placement can be lateral, medial, or both lateral and medial. For certain local applications the adhesive bioerodible drug delivery system of the present invention can be used to treat diseases affecting the eyelids. For delivery into the eye, placement on the bulbar conjunctiva is preferred. Exceptions for bulbar placement could be placement on the limbus or cornea, especially for purposes of wound healing or other indications related to tissue regeneration or local infection.

The present invention provides a mucoadhesive film that includes: a water-soluble bioadhesive layer to be placed in contact with an ocular surface, the bioadhesive layer including one or more bioadhesive polymers and/or one or more film-forming, water-soluble polymers; a water-soluble non-adhesive backing layer that includes one or more water-soluble, film-forming, pharmaceutically acceptable polymers; and one or more pharmaceuticals associated with the bioadhesive layer, associated with the non-adhesive layer, or associated with both the bioadhesive and non-adhesive layers; wherein the mucoadhesive film is compatible with ocular surfaces; the mucoadhesive film adheres to ocular surfaces; the mucoadhesive film is flexible; and the mucoadhesive film is water-soluble, biodegradable, and bioerodible in tear fluids.

The present invention also provides a method for treating a wound on an ocular surface of a mammal including contacting the ocular surface of the mammal afflicted with the wound with a mucoadhesive film of the present invention.

The present invention also provides a method for delivering a pharmaceutical to an ocular surface of a mammal including contacting the ocular surface of the mammal with a mucoadhesive film of the present invention.

The present invention also provides a method for treating a mammal suffering from a migraine headache, the method including contacting an ocular surface of the mammal afflicted with the migraine headache with a mucoadhesive film of the present invention.

The present invention also provides a method for treating a mammal suffering from diabetic retinopathy, the method including contacting an ocular surface of the mammal afflicted with the diabetic retinopathy with a mucoadhesive film of the present invention.

The present invention also provides a method for treating a mammal suffering from muscular degeneration, the method including contacting an ocular surface of the mammal afflicted with the muscular degeneration with a mucoadhesive film of the present invention.

The present invention also provides a method for treating a mammal suffering from uveitis, the method including contacting an ocular surface of the mammal afflicted with the uveitis with a mucoadhesive film-of the present invention.

The present invention also provides a method for treating a mammal suffering from herpetic conjunctivitis, the method including contacting an ocular surface of the mammal afflicted with the herpetic conjunctivitis with a mucoadhesive film of the present invention.

The present invention also provides a method for treating a mammal suffering from blepharitis, the method including contacting an ocular surface of the mammal afflicted with the blepharitis with a mucoadhesive film of the present invention.

The present invention also provides a method for treating a mammal suffering from macular degeneration (e.g., age-related macular degeneration), the method including contacting an ocular surface of the mammal afflicted with the macular degeneration with a mucoadhesive film of the present invention.

The present invention also provides a method for locally delivering one or more pharmaceuticals to an ocular region of a mammal, the method including contacting the ocular surface of the mammal with a mucoadhesive film of the present invention.

The present invention also provides a method for systemically delivering one or more pharmaceuticals to a mammal via an ocular surface, the method including contacting the ocular surface of the mammal with a mucoadhesive film of the present invention.

The present invention also provides a mucoadhesive film that includes: a water-soluble bioadhesive layer to be placed in contact with an ocular surface, the bioadhesive layer including one or more bioadhesive polymers and/or one or more film-forming, water-soluble polymers; a water-soluble non-adhesive backing layer that includes one or more water-soluble, film-forming, pharmaceutically acceptable polymers; and one or more pharmaceuticals associated with the bioadhesive layer, associated with the non-adhesive layer, or associated with both the bioadhesive and non-adhesive layers; wherein the mucoadhesive film is compatible with ocular surfaces; the mucoadhesive film adheres to ocular surfaces; the mucoadhesive film is flexible; and the mucoadhesive film is water-soluble, biodegradable, and bioerodible in tear fluids; for use in medical therapy.

The present invention also provides the use of a mucoadhesive film that includes: a water-soluble bioadhesive layer to be placed in contact with an ocular surface, the bioadhesive layer including one or more bioadhesive polymers and/or one or more film-forming, water-soluble polymers; a water-soluble non-adhesive backing layer that includes one or more water-soluble, film-forming, pharmaceutically acceptable polymers; and one or more pharmaceuticals associated with the bioadhesive layer, associated with the non-adhesive layer, or associated with both the bioadhesive and non-adhesive layers; wherein the mucoadhesive film is compatible with ocular surfaces; the mucoadhesive film adheres to ocular surfaces; the mucoadhesive film is flexible; and the mucoadhesive film is water-soluble, biodegradable, and bioerodible in tear fluids; for the manufacture of a medicament for treating a wound on an ocular surface of a mammal, for delivering a pharmaceutical to an ocular surface of a mammal, for treating a mammal suffering from a migraine headache, for treating a mammal suffering from diabetic retinopathy, for treating a mammal suffering from muscular degeneration, for treating a mammal suffering from uveitis, for treating a mammal suffering from herpetic conjunctivitis, for treating a mammal suffering from blepharitis, for treating a mammal suffering from macular degeneration (e.g., age-related macular degeneration), for locally delivering one or more pharmaceuticals to an ocular region of a mammal, and/or for systemically delivering one or more pharmaceuticals to a mammal via an ocular surface.

The present invention also provides a kit that includes the mucoadhesive film of the present invention and instructions for using the mucoadhesive film.

### BRIEF DESCRIPTION OF THE DRAWINGS

Embodiments of the invention may be best understood by referring to the following description and accompanying drawings which illustrate such embodiments. The numbering scheme for the Figures included herein are such that the leading number for a given reference number in a Figure is associated with the number of the Figure. Reference numbers are the same for those elements that are the same across different Figures. For example, ocular regions and ocular surfaces, such as the lacrimal ducts (110) can be located in FIG. 1. However, reference numbers are the same for those elements that are the same across different Figures. In the drawings:

FIG. 1 illustrates ocular regions and ocular surfaces useful in the present invention.

FIG. 2 illustrates ocular regions and ocular surfaces useful in the present invention.

FIG. 3 illustrates ocular regions and ocular surfaces useful in the present invention.

FIG. 4 illustrates mucosal regions and mucosal surfaces useful in the present invention.

FIG. 5 illustrates a mucoadhesive film of the present invention, in use, on an ocular surface.

### DETAILED DESCRIPTION OF THE INVENTION

References in the specification to “one embodiment”, “an embodiment”, “an example embodiment”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

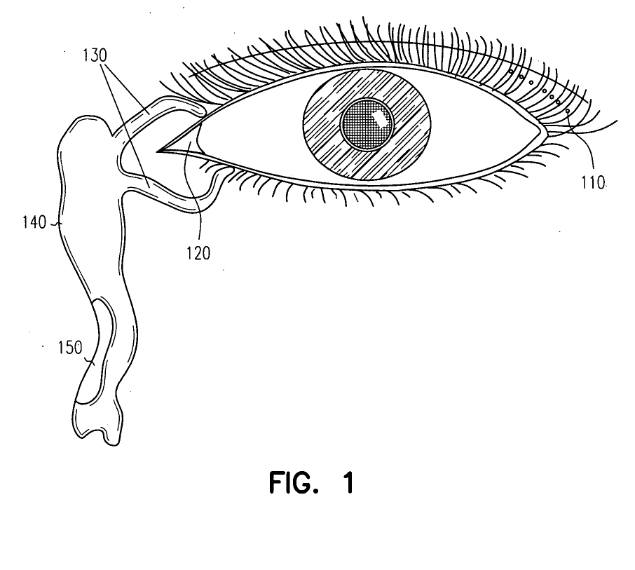

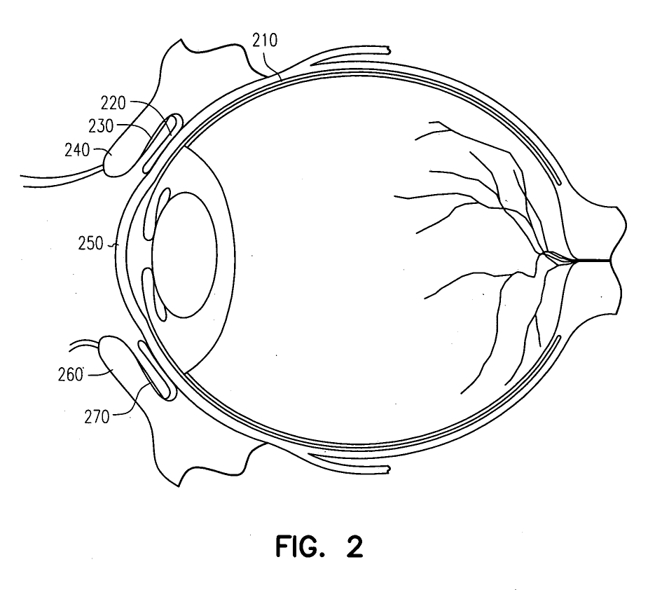

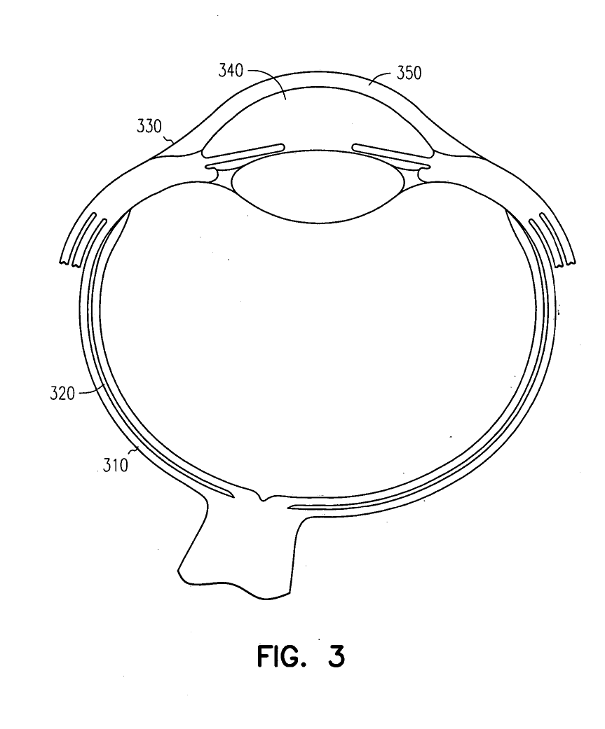

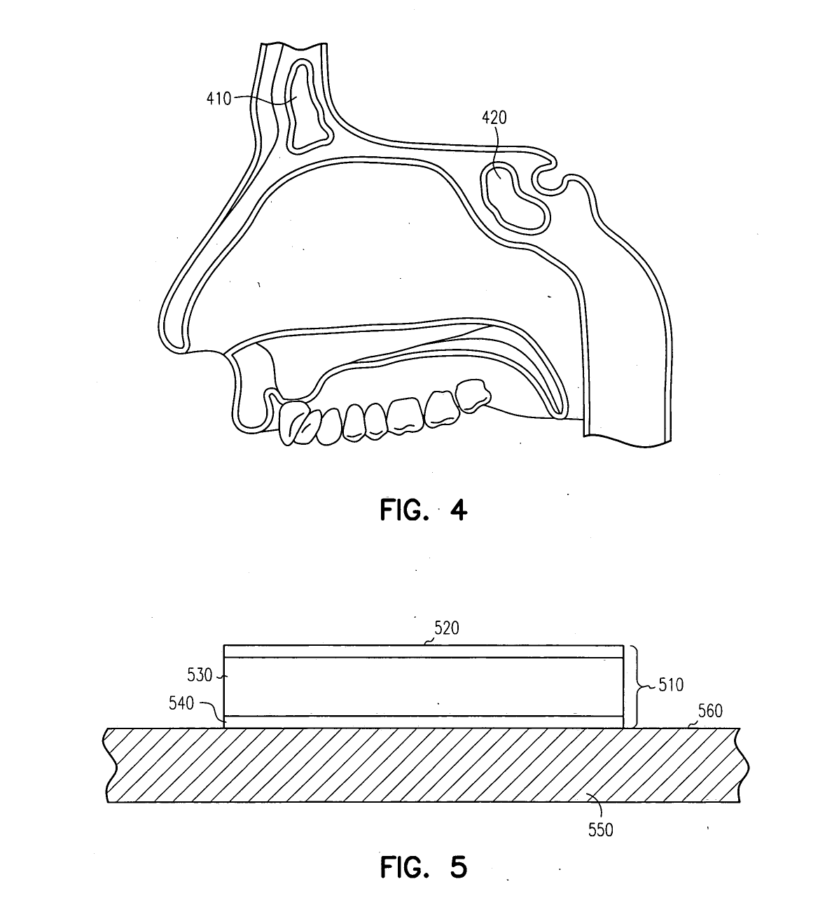

Referring to FIGS. 1-5, the present invention provides a mucoadhesive film (510) that includes: a water-soluble bioadhesive layer (530) to be placed in contact with an ocular surface (560), the bioadhesive layer including one or more bioadhesive polymers and/or one or more film-forming, water-soluble polymers; a water-soluble non-adhesive backing layer (540) that includes one or more water-soluble, film-forming, pharmaceutically acceptable polymers; and one or more pharmaceuticals associated with the bioadhesive layer, associated with the non-adhesive layer, or associated with both the bioadhesive and non-adhesive layers; wherein the mucoadhesive film (510) is compatible with ocular surfaces (560); the mucoadhesive film (510) adheres to ocular surfaces (560); the mucoadhesive film (510) is flexible; and the mucoadhesive film (510) is water-soluble, biodegradable, and bioerodible in tear fluids.

Since the mucoadhesive film (510) can be placed on an ocular surface (560), the mucoadhesive film (510) will preferably be made from materials generally regarded as safe (“GRAS-certified), or national formulary (“NF-certified”), and therefore acceptable and safe for ophthalmic use.

As used herein, “ocular” or “ocular region” (550) refers to the eye, surrounding tissues, and to bodily fluids in the region of the eye. Specifically, the term includes the cornea (350) or (250), the sclera (310) or (210), the uvea (320), the conjunctiva (330) (e.g., bulbar conjunctiva (220), palpebral conjunctiva (230), and tarsal conjunctiva (270)), anterior chamber (340), lacrimal sac, lacrimal canals (130), lacrimal ducts (110), medial canthus (120), nasolacrimal duct (150), and the eyelids (e.g., upper eyelid (240) and lower eyelid (260)). Additionally, the term includes the inner surface of the eye (conjunctiva overlying the sclera (310) or (210)), and the inner surface of the eyelids (palpepral conjunctiva).

As used herein, “conjunctiva” refers to the mucous membrane lining the inner surfaces of the eyelids and anterior part of the sclera (310) or (210). The “palpebral conjunctiva” lines the inner surface of the eyelids and is thick, opaque, and highly vascular. The “bulbar conjunctiva” is loosely connected, thin, and transparent, covering the sclera (310) or (210) of the anterior third of the eye.

As used herein, “cornea” refers to the convex, transparent anterior part of the eye, comprising one sixth of the outermost tunic of the eye bulb. It allows light to pass through it to the lens. The cornea (350) or (250) is a fibrous structure with five layers: the anterior corneal epithelium, continuous with that of the conjunctiva; the anterior limiting layer (Bowman's membrane); the substantial propria; the posterior limiting layer (Descemet's membrane); and the endothelium of the anterior chamber (340) (keratoderma). It is dense, uniform in thickness, and nonvascular, and it projects like a dome beyond the sclera (310) or (210), which forms the other five sixths of the eye's outermost tunic. The degree of corneal curvature varies among different individuals and in the same person at different ages; the curvature is more pronounced in youth than in advanced age.

As used herein, “eye” refers to one of a pair of organs of sight, contained in a bony orbit at the front of the skull, embedded in orbital fat, and innervated by four cranial nerves: optic, oculomotor, trochlear, and abducens. Associated with the eye are certain accessory structures, such as the muscles, the fasciae, the eyebrow, the eyelids, the conjunctiva (330), and the lacrimal gland. The bulb of the eye is composed of segments of two spheres with nearly parallel axes that constitute the outside tunic and one of three fibrous layers enclosing two internal cavities separated by the crystalline lens. The smaller cavity anterior to the lens is divided by the iris into two chambers, both filled with aqueous humor. The posterior cavity is larger than the anterior cavity and contains the jellylike vitreous body that is divided by the hyaloid canal. The outside tunic of the bulb consists of the transparent cornea anteriorly, constituting one fifth of the tunic, and the opaque sclera posteriorly, constituting five sixths of the tunic. The intermediate vascular, pigmented tunic consists of the choroid, the ciliary body, and the iris. The internal tunic of nervous tissue is the retina. Light waves passing through the lens strike a layer of rods and cones in the retina, creating impulses that are transmitted by the optic nerve to the brain. The transverse and the anteroposterior diameters of the eye bulb are slightly greater than the vertical diameter; the bulb in women is usually smaller than the bulb in men. Eye movement is controlled by six muscles: the superior and inferior oblique muscles and the superior, inferior, medial, and lateral rectus muscles. Also called bulbus oculi, eyeball.

As used herein, “eyelid” refers to a movable fold of thin skin over the eye, with eyelashes and ciliary and meibomian glands along its margin. It consists of loose connective tissue containing a thin plate of fibrous tissue lined with mucous membrane (conjunctiva). The orbicularis oculi muscle and the oculomotor nerve control the opening and closing of the eyelid. The upper and lower eyelids are separated by the palpebral fissure. Also called palpebra.

As used herein, “canthus” refers to a corner of the eye, the angle at the medial and the lateral margins of the eyelids. The medial canthus (120) opens into a small space containing the opening to a lacrimal duct. Also called palpebral commissure.

As used herein, “mucus” refers to the viscous, slippery secretions of mucous membranes and glands, containing mucin, white blood cells, water, inorganic salts, and exfoliated cells.

As used herein, “nasal sinus” refers to any one of the numerous cavities in various bones of the skull, lined with ciliated mucous membrane continuous with that of the nasal cavity. The membrane is very sensitive; easily irritated, it may cause swelling that blocks the sinuses. The nasal sinus can include, e.g., the frontal sinus (410) or the spheroidal sinus (420).

As used herein, “lacrimal” refers to tears.

As used herein, “lacrimal duct” refers to one of a pair of channels through which tears pass from the lacrimal lake to the lacrimal sac of each eye. Also called lacrimal canaliculus.

As used herein, “palpebral conjunctiva” refers to the mucous membrane lining the inner surfaces of the eyelids and anterior part of the sclera (310) or (210). The “palpebral conjunctiva” lines the inner surface of the eyelids and is thick, opaque, and highly vascular. The “bulbar conjunctiva” is loosely connected, thin, and transparent, covering the sclera (310) or (210) of the anterior third of the eye.

As used herein, “retina” refers to a 10-layered, delicate nervous tissue membrane of the eye, continuous with the optic nerve, that receives images of external objects and transmits visual impulses through the optic nerve to the brain. The retina is soft and semitransparent and contains rhodopsin. It consists of the outer pigmented layer and the nine-layered retina proper. These nine layers, starting with the most internal, are the internal limiting membrane, the stratum opticum, the ganglion cell layer, the inner plexiform layer, the inner nuclear layer, the outer plexiform layer, the outer nuclear layer, the external limiting membrane, and the layer of rods and cones. The outer surface of the retina is in contact with the choroid; the inner surface with the vitreous body. The retina is thinner anteriorly, where it extends nearly as far as the ciliary body, and thicker posteriorly, except for a thin spot in the exact center of the posterior surface where focus is best. The photoreceptors end anteriorly in the jagged ora serrata at the ciliary body, but the membrane of the retina extends over the back of the ciliary processes and the iris. The retina becomes clouded and opaque if exposed to direct sunlight. See also Jacob's membrane, macula, optic disc.

As used herein, “retinochoroid” refers to an inflammation of the retina and choroid coat of the eye.

As used herein, “sclera” refers to the tough inelastic opaque membrane covering the posterior five sixths of the eyebulb. It maintains the size and form of the bulb and attaches to muscles that move the bulb. Posteriorly it is pierced by the optic nerve and, with the transparent cornea, makes up the outermost of three tunics covering the eyebulb.

As used herein, “sinus” refers to a cavity or channel, such as a cavity-within a bone, a dilated channel for venous blood, or one permitting the escape of purulent material.

As used herein, “tarsal gland” refers to any one of numerous modified sebaceous glands on the inner surfaces of the eyelids. Acute localized bacterial infection of a tarsal gland may cause a sty or a chalazion.

As used herein, “tears” refers to a watery saline or alkaline fluid secreted by the lacrimal glands to moisten the conjunctiva.

As used herein, “uvea” refers to the fibrous tunic beneath the sclera (310) or (210) that includes the iris, the ciliary body, and the choroid of the eye.

As used herein, “vasculature” refers to the distribution of blood vessels in an organ or tissue.

As used herein, “treat” or “treating” refers to: (i) preventing a pathologic condition from occurring (e.g. prophylaxis) or symptoms related to the same; (ii) inhibiting the pathologic condition or arresting its development or symptoms related to the same; or (iii) relieving the pathologic condition or symptoms related to the same.

Water-Soluble Bioadhesive Layer (530)

The water-soluble bioadhesive layer (530) can adhere to the ocular surface (560) (e.g., conjunctiva or cornea (350) or (250) surface) of a mammal. The water-soluble bioadhesive layer (530) is generally water-soluble and can be made from film-forming water-soluble polymer(s) and bioadhesive polymer(s). More specifically, the bioadhesive layer can include at least one film-forming water-soluble polymer (the “film-forming polymer”) and/or at least one pharmacologically acceptable polymer known for its bioadhesive capabilities (the “bioadhesive polymer”). More specifically, the bioadhesive layer can include only one polymer, e.g., hydroxyethylmethyl cellulose (HEMC) that acts as both the bioadhesive and film-former. Alternatively, the water-soluble bioadhesive layer (530) can include film-forming water-soluble polymer(s) and water-soluble plasticizer(s), such as glycerin and/or polyethylene glycol (PEG).

Film-Forming, Water-Soluble Polymer

The film-forming water-soluble polymer(s) of the bioadhesive layer can be cellulose derivatives. Such film-forming water-soluble polymer(s) can include hydroxyethyl cellulose (HEC), hydroxypropyl cellulose (HPC), hydroxypropylmethyl cellulose (HPMC), hydroxyethylmethyl cellulose (HEMC), or a combination thereof. Similar film-forming water-soluble polymer(s) can also be used. The film-forming water-soluble polymer(s) can optionally be crosslinked and/or plasticized in order to alter its dissolution kinetics.

Bioadhesive Polymer

The bioadhesive polymer of the water-soluble bioadhesive layer (530) can include polyacrylic acid (PAA), which can optionally be partially crosslinked, sodium carboxymethyl cellulose (NaCMC), hydroxypropylmethyl cellulose (HPMC), polyvinylpyrrolidone (PVP), or combinations thereof. These bioadhesive polymers are preferred because they have good and instantaneous mucoadhesive properties in a dry, film state. Other bioadhesive polymers having similarly useful properties and that known to one of skill in the art can also be used.

The simultaneous use of PAA with some grades of PVP can result in the precipitation of one or both components. This precipitation may not be desirable, especially when attempting to form a homogenous layer. Moreover, such precipitation may slightly alter the overall adhesive properties of the mucoadhesive film (510). It is appreciated that one of skill in the art can recognize these problems and avoid use of those grades of PVP with PAA.

Water-Soluble Non-Adhesive Backing Layer (540)

The water-soluble non-adhesive backing layer (540) is also water-soluble and includes pharmaceutically acceptable, water-soluble, film-forming polymer(s). The non-adhesive backing layer will dissolve after application of the mucoadhesive film (510) to a conjunctiva (330) surface of a mammal. More specifically, the water-soluble non-adhesive backing layer (540) will typically dissolve before the water-soluble bioadhesive layer (530) dissolves.

The water-soluble non-adhesive backing layer (540) protects the water-soluble bioadhesive layer (530). Dissolution of the water-soluble non-adhesive backing layer (540) primarily controls the residence time of the mucoadhesive film (510) of the present invention after application to the conjunctiva (330) and promotes unidirectional delivery across the target conjunctiva (330).

Water-Soluble, Film-Forming, Pharmaceutically Acceptable Polymer

The water-soluble non-adhesive backing layer (540) includes water-soluble, film-forming pharmaceutically acceptable polymer(s) such as, but not limited to, hydroxyethyl cellulose (HEC), hydroxypropyl cellulose (HPC), hydroxypropylmethyl cellulose (HPMC), hydroxyethylmethyl cellulose (HEMC), polyvinyl alcohol (PVA), polyethylene glycol (PEG), polyethylene oxide (PEO), ethylene oxide-propylene oxide co-polymers, or a combination thereof. The water-soluble non-adhesive backing layer (540) component can optionally be crosslinked. In one embodiment, the water-soluble non-adhesive backing layer (540) includes hydroxyethyl cellulose and hydroxypropyl cellulose. The water-soluble non-adhesive backing layer (540) can function as a slippery surface, to avoid “double-stick” to bulbar and palpebral conjunctiva (230).

Combinations of different polymers or similar polymers with definite molecular weight characteristics can be used in order to achieve preferred film forming capabilities, mechanical properties, and kinetics of dissolution.

Pharmaceutical

The pharmaceutical(s) can be located throughout the water-soluble bioadhesive layer (530), throughout the water-soluble, non-adhesive backing layer, or throughout both the water-soluble bioadhesive layer (530) and the water-soluble, non-adhesive backing layer. Specifically, the pharmaceutical(s) can be located uniformly throughout the water-soluble bioadhesive layer (530), uniformly throughout the water-soluble, non-adhesive backing layer, or uniformly throughout both the water-soluble bioadhesive layer (530) and the water-soluble, non-adhesive backing layer. Alternatively, the pharmaceutical(s) can be located near the center of the water-soluble bioadhesive layer (530) and the periphery of the water-soluble bioadhesive layer (530) can adhere to an ocular surface (560) (e.g., conjunctiva (330) or cornea (350) or (250) surface) of a mammal. After application to the ocular surface (560) of a mammal, the mucoadhesive film (510) can provide sustained delivery of the pharmaceutical(s).

The pharmaceutical can be suitable for local delivery in the eye. Alternatively, the pharmaceutical can be suitable for systemic delivery via the eye.

Compositions that include the pharmaceutical(s) that are incorporated into the mucoadhesive film (510) of the present invention can be a liquid, solid, suspension, molten or powder composition when deposited onto either layer of the mucoadhesive film (510). Such compositions can include any pharmaceutical(s) selected by one of skill in the art. The composition(s) can be deposited onto either layer more than once, for example, the composition can be deposited onto either layer between about 1 to about 10 times. The composition loaded into the mucoadhesive film (510) of the present invention can include any excipient selected by one of skill in the art. For example, polymeric and nonpolymeric viscosity-building agents, polymeric and nonpolymeric hydrophilicity agents, and combinations thereof, can be employed as excipients. The addition of the pharmaceutical after film formation is called “post-loading.”

The pharmaceutical can be added when the polymers are mixed prior to coating and subsequent drying to form the film. The addition of the pharmaceutical prior to film formation is called preloading. The pharmaceutical can be either dissolved or dispersed in a liquid or gel. The liquid or gel also includes the polymer(s) and other excipients. This liquid or gel is then processed to form a flexible layer of the multilayer, bioerodible, mucoadhesive film (510).

The pharmaceutical can include a single pharmaceutical or a combination of pharmaceuticals. Examples of categories of pharmaceuticals that can be used, either alone or in combination include: adrenergic agent; adrenocortical steroid; adrenocortical suppressant; alcohol deterrent; aldosterone antagonist; amino acid; ammonia detoxicant; anabolic; analeptic; analgesic; androgen; anti-angiogenic; anesthesia, adjunt to; anesthetic; anorectic; antagonist; anterior pituitary suppressant; anthelmintic; antiacne agent; anti-adrenergic; anti-allergic; anti-amebic; anti-androgen; anti-anemic antianginal; anti-anxiety; anti-arthritic; anti-asthmatic; anti-atherosclerotic; antibacterial; anticholelithic; anticholelithogenic; anticholinergic; anticoagulant; anticoccidal; anticonvulsant; antidepressant; antidiabetic; antidiarrheal; antidiurietic; antidote; anti-emetic; anti-epileptic; anti-estrogen; antifibronolytic; antifungal; antiglaucoma agent; antihemophilic; antihermorrhagic; antihistamine; antihyperlipidemia; antihyperlipoproteinemic; antihypertensive; antihypotensive; anti-infective; anti-infective, topical; anti-inflammatory; antikeratinizing agent; antimalarial; antimicrobial; antimigraine; antimycotic, antinausant, antineoplastic, antineutropenic, antiobessional agent; antiparasitic; antiparkinsonian; antiperistaltic, antipneumocystic; antiproliferative; antiprostatic hypertrophy; antiprotozoal; antipruritic; antipsychotic; antirheumatic; antischistosomal; antiseborrheic; antisecretory; antispasmodic; antithrombotic; antitussive; anti-ulcerative; anti-urolithic; antiviral; appetite suppressant; benign prostatic hyperplasia therapy agent; blood glucose regulator; bone resorption inhibitor; bronchodilator; carbonic anhydrase inhibitor; cardiac depressant; cardioprotectant; cardiotonic; cardiovascular agent; choleretic; cholinergic; cholinergie diagnostic aid; diuretic; dopaminergic agent; ectoparasiticide; emetic; enxzyme inhibitor;, estrogen; fibrinolytic; flourescent agent, free oxygen radical scavenger; gastrointestinal motility effector; glucocorticoid; gonad-stimulating principle; hair growth stimulant; hemostatic; histamine H2 receptor antagonist; hormone; hypocholesterolemic; hypoglycemic; hypolipidemic; hypotensive; imaging agent; immunizing agent; immunomodulator; immunoregulator; immunostimulant; immunosuppressant; impotence therapy; inhibitor; keratolytic; LNRN agonist; liver disorder treatment; luteolysin; memory adjuvant; mental performance enhancer; mood regulator; mucolytic; mucosal protective agent; mydriatic; nasal decongestant; neuromuscular blocking agent; neuroprotective; NMDA antagonist; non-hormonal sterol derivative; oxytocic; plasminogen activator; platelet activating factor antagonist; platelet aggregaton inhibitor; post-stroke and post-head trauma treatment; potentiator; progestin; prostaglandin; prostate growth inhibitor; prothyrotropin; psychotropic; radioactive agent; regulator; relaxant; repartitioning agent; scabicide; sclerosing agent; sedative; sedative-hypnotic; selective adenosine A1 antagonist; serotonin antagonist; serotinin inhibitor; serotinin receptor antagonist; steroid; stimulant; suppressant; symptomatic multiple sclerosis; synergist; thyroid hormone; thyroid inhibitor; thyromimetic; tranquilizer; treatment of amyotrophic laterial sclerosis; treatment of cerebral ischemia; treatment of Paget's disease; treatment of unstable angina; uricosuric; vasoconstrictor; vasodilator; vulnerary; wound healing agent; and zxanthine oxidase inhibitor.

Specific pharmaceuticals that are examples of the classes of pharmaceuticals disclosed above include, but are not limied to, Acebutolol; Acebutolol; Acyclovir; Albuterol; Alfentanil; Almotriptan; Alprazlam; Amiodarone; Amlexanox; Amphotericin B; Anecortave Acetate; Atorvastatin; Atropine; Auranofin; Aurothioglucose; Benazepril; Bicalutamide; Bretylium; Brifentanil; Bromocriptine; Buprenorphine; Butorphanol; Buspirone; Calcitonin; Candesartan; Carfentanil; Carvedilol; Chlorpheniramine; Chlorothiazide; Chlorphentermine; Chlorpromazine; Clindamycin; Clonidine; Codeine; Cyclosporine; Desipramine; Desmopressin; Dexamethasone; Diazepam; Diclofenac; Digoxin; Digydrocodeine; Dolasetron; Dopamine; Doxepin; Doxycycline; Dronabinol; Droperidol; Dyclonine; Eletriptan; Enalapril; Enoxaparin; Ephedrine; Epinephrine; Ergotamine; Etomidate; Famotidine; Felodipine; Fentanyl; Fexofenadine; Fluconazole; Fluoxetine; Fluphenazine; Flurbiprofen; Fluvastatin; Fluvoxamine; Frovatriptan; Furosemide; Ganciclovir; Gold sodium thiomalate; Granisetron; Griseofulvin; Haloperidol; Hepatitis B Virus Vaccine; Hydralazine; Hydromorphone; Insulin; Ipratropium; Isradipine; Isosorbide Dinitrate; Ketamine; Ketorolac; Labetalol; Levorphanol; Lisinopril; Loratadine; Lorazepam; Losartan; Lovastatin; Melatonin; Methyldopa; Methylphenidate; Metoprolol; Midazolam; Mirtazapine; Morhpine; Nadolol; Nalbuphine; Naloxone; Naltrexone; Naratriptan; Neostgmine; Nicardipine; Nifedipine; Norepinephrine; Nortriptyline; Octreotide and analogues thereof; Olanzapine; Omeprazole; Ondansetron; Oxybutynin; Oxycodone; Oxymorphone; Oxytocin; Phenylephrine; Phenylpropanolaimine; Phenytoin; Pimozide; Pioglitazone; Piroxicam; Pravastatin; Prazosin; Prochlorperazine; Propafenone; Prochlorperazine; Propiomazine; Propofol; Propranolol; Pseudoephedrine; Pyridostigmine; Quetiapine; Raloxifene; Remifentanil; rhuFab V2; Rofecoxib; Repaglinide; Risperidone; Rizatriptan; Ropinirole; Somatostatin and analogues thereof; Scopolamine; Selegiline; Sertraline; Sildenafil; Simvastatin; Sirolimus; Spironolactone; Sufentanil; Sumatriptan; Tacrolimus; Tamoxifen; Terbinafine; Terbutaline; Testosterone; Tetanus toxoid; THC Tolterodine; Triamterene; Triazolam; Tricetamide; Valsartan; Venlafaxine; Verapamil; Visudyne; Zaleplon; Zanamivir; Zafirlukast; Zolmitriptan; and Zolpidem.

The amount of pharmaceutical to be placed with the composition depends on the desired treatment dosage to be administered, although typically, the pharmaceutical component will be present in about 0.001% to about 50% by weight of the mucoadhesive film (510), and more specifically between about 0.005 and about 35% by weight.

In one embodiment, the mucoadhesive film (510) of the present invention can include an antimigraine medication as the pharmaceutical. The antimigraine medication can be located in the water-soluble bioadhesive layer (530). The water-soluble bioadhesive layer (530) can be placed adherent to the palpebral conjunctiva (230). The antimigraine medication can include, e.g., naratriptan, zolmitriptan, rizatriptan, frovatriptan, octreatide, sumatriptan or other “triptan” pharmaceutical. The mucoadhesive film (510) has the advantages of rapid plasma levels and avoidance of first-pass metabolism.

In another embodiment, the mucoadhesive film (510) of the present invention can include an antiangiogenic agent as the pharmaceutical. The antiangiogenic agent can be located in the water-soluble bioadhesive layer (530). The mucoadhesive film (510) can deliver to the retinochoroid the antiangiogenic agent, to effectively treat patients with diabetic retinopathy or macular degeneration.

In another embodiment, the mucoadhesive film (510) of the present invention can include an immunosuppressive as the pharmaceutical, to effectively treat patients with uveitis.

In another embodiment, the mucoadhesive film (510) of the present invention can include an immunosuppresive or anti-inflammatory agent as the pharmaceutical. The mucoadhesive film (510) can locally deliver to the tarsal conjunctiva (270) the immunosuppresive or the anti-inflammatory agent, to effectively treat vernal keratoconjunctivitis.

In another embodiment, the mucoadhesive film (510) of the present invention can include a would-healing medication as the pharmaceutical. The mucoadhesive film (510) would effectively hold the pharmaceutical in direct contact with a corneal wound.

In another embodiment, the mucoadhesive film (510) of the present invention can include an antiviral agent, an antibiotic agent, an antifungal agent, or a combination thereof. The mucoadhesive film (510) would effectively treat infectious diseases (e.g., bacterial, viral, or fungal).

In another embodiment, the mucoadhesive film (510) of the present invention can include an antiviral agent. The mucoadhesive film (510) would deliver the antiviral agent to the cornea (350) or (250), thereby effectively treating patients afflicted with herpetic conjunctivitis or blepharitis.

Lubrication Layer (520)

A non-water soluble lubrication layer (520) can optionally be applied to the water-soluble, non-adhesive backing layer. This would be in the form of a non-continuous film of a silicon or hydrocarbon such as petrolatum. This lubrication layer (520) would provide improved comfort until the delivery system fully hydrates.

Cross-Linking Agent

In order to modify the water dissolution kinetics of the backing layer without resulting in a non-water soluble material, partial and limited crosslinking can optionally be used. More specifically, when employed, the cross-linking agent, upon cross-linking the backing layer, will effectively decrease the disintegration rate and lengthen the residence time of the mucoadhesive film (510). Crosslinking agents known in the art are appropriate for use in the invention and can include, e.g., glyoxal, propylene glycol, glycerol, dihydroxy-polyethylene glycol of different sizes, and butylene glycol. The amount of crosslinking agent used can vary, depending on the particular polymers and crosslinking agent employed, but should not exceed 5% molar equivalent of the water-soluble, film-forming pharmaceutically acceptable polymer(s), and preferably includes 0% to about 3% molar equivalent of the water-soluble, film-forming pharmaceutically acceptable polymer(s). Dissolution characteristics can be adjusted to modify the residence time and the release profile of a pharmaceutical(s) when included in the water-soluble, non-adhesive backing layer.

Physical Dimension

The thickness of the mucoadhesive film (510) of the present invention may vary, depending on the thickness of each of the layers. Typically, the bilayer thickness ranges from about 0.01 mm to about 1 mm, and more specifically, from about 0.05 mm to about 0.5 mm. The thickness of each layer can vary from about 10% to about 90% of the overall thickness of the bilayer mucoadhesive film (510), and specifically can vary from about 30% to about 60% of the overall thickness of the bilayer mucoadhesive film (510). Thus, the preferred thickness of each layer can vary from about 0.005 mm to about 1.0 mm, and more specifically from about 0.01 mm to about 0.5 mm.

Pharmaceutically acceptable dissolution-rate-modifying agent, pharmaceutically acceptable disintegration aid (e.g., polyethylene glycol, dextran, polycarbophil, carboxymethyl cellulose, or poloxamers), pharmaceutically acceptable plasticizer, pharmaceutically acceptable coloring agent (e.g., FD&C Blue #1), pharmaceutically acceptable opaquifier (e.g., titanium dioxide), pharmaceutically acceptable anti-oxidant (e.g., tocopherol acetate), pharmaceutically acceptable film forming enhancer (e.g., polyvinyl alcohol or polyvinyl pyrrolidone), pharmaceutically acceptable preservative, or a combination thereof can optionally be included in the mucoadhesive film (510). Preferably, these components include no more than about 1% of the final weight of the mucoadhesive film (510), but the amount may vary depending on the pharmaceutical(s) or other components of the mucoadhesive film (510). One of skill in the art can readily achieve appropriate concentrations of these components.

Plasticizer

The mucoadhesive film (510) can optionally include one or more plasticizers, to soften, increase the toughness, increase the flexibility, improve the molding properties, and/or otherwise modify the properties of the mucoadhesive film (510). Plasticizers for use in the present invention can include, e.g., those plasticizers having a relatively low volatility such as glycerin, propylene glycol, sorbitol, ethylene glycol, diethylene glycol, triethylene glycol, propylene glycol, polypropylene glycol, dipropylene glycol, butylenes glycol, diglycerol, polyethylene glycol (e.g., low molecular weight PEG's), oleyl alcohol, cetyl alcohol, cetostearyl alcohol, and other pharmaceutical-grade alcohols and diols having boiling points above about 100° C. at standard atmospheric pressure (1 atm.). Additional plasticizers include, e.g., polysorbate 80, triethyl titrate, acetyl triethyl titrate, and tributyl titrate. Additional suitable plasticizers include, e.g., diethyl phthalate, butyl phthalyl butyl glycolate, glycerin triacetin, and tributyrin. Additional suitable plasticizers include, e.g., pharmaceutical grade hydrocarbons such as mineral oil (e.g., light mineral oil) and petrolatum. Additional suitable plasticizers include, e.g., triglycerides such as medium-chain triglyceride, soybean oil, safflower oil, peanut oil, and other pharmaceutical grade triglycerides. Additional suitable plasticizers include, e.g., PEGylated triglycerides such as Labrifil®, Labrasol® and PEG-4 beeswax. Additional suitable plasticizers include, e.g., lanolin. Additional suitable plasticizers include, e.g., polyethylene oxide (PEO) and other polyethylene glycols. Additional suitable plasticizers include, e.g., hydrophobic esters such as ethyl oleate, isopropyl myristate, isopropyl palmitate, cetyl ester wax, glyceryl monolaurate, and glyceryl monostearate. Additional suitable plasticizers include, e.g., those plasticizers disclosed in U.S. Pat. No. 5,700,478.

Disintegration Aids

One or more disintegration aids can optionally be employed to increase the disintegration rate and shorten the residence time of the mucoadhesive film (510) of the present invention. Disintegration aids useful in the present invention include, e.g., hydrophilic compounds such as water, methanol, ethanol, or low alkyl alcohols such as isopropyl alcohol, acetone, methyl ethyl acetone, alone or in combination. Specific disintegration aids include those having less volatility such as glycerin, propylene glycol, and polyethylene glycol.

Dissolution-Rate-Modifying Agent

One or more dissolution-rate-modifying agents can optionally be employed to decrease the disintegration rate and lengthen the residence time of the mucoadhesive film (510) of the present invention. Dissolution-rate-modifying agents useful in the present invention include, e.g., hydrophobic compounds such as heptane, and dichloroethane, alone or in combination.

Peelable Sheet

In the mucoadhesive film (510) of the present invention, a sheet including the mucoadhesive film (510) may be provided on one side surface and/or the peelable sheet may be provided on one side or both side surface(s), or the sheet may be provided on one side surface and the peelable sheet is provided on another side surface, in view of protection of the adhesive sheet and convenience in handling upon application to human conjunctiva (330).

The peelable sheet is not particularly restricted, so long as the sheet is a film having a high peelability. Namely, examples of the material of the peelable sheet include a film including a resin selected from the group polyethylene, polyethyleneterephthalate, polypropylene, polystyrene, polyvinylchloride, polyvinyl alcohol and Saran; polyethylene-coated wood free paper; polyolefin-coated glassine paper; paper, aluminum thin film or the above resins, surface-treated with silicone. Among these, a film including resin of polyethylene or Saran is preferred. The thickness of the peelable sheet can be from about 1 μm to about 500 μm, more specifically from about 5 μm to about 200 μm, and more specifically from about 20 μm to about 100 μm, in viewpoint of handling and cost.

Packaging

The mucoadhesive film (510) of the present invention can be packed in an airtight package material and stored to prevent deterioration in qualities due to moisture. Specific examples of the airtight package material include, e.g., cellophane, moistureproof cellophane, polypropylene, nylon, polyester, vinylidene chloride, vinyl chloride, polycarbonate, low-density polyethylene, high-density polyethylene, linear low-density polyethylene, ionomer, polyvinyl alcohol, ethylene/vinyl acetate copolymer, ethylene/acrylic acid copolymer, ethylene/ethyl acrylate copolymer, polymethylpentene, polystyrene, aluminum foil, etc. Among these materials, films having polypropylene, vinylidene chloride, low-density polyethylene, high-density polyethylene, linear low-density polyethylene or aluminum foil laminated thereon are particularly preferable due to their excellent barrier properties to vapor permeation. Regarding the barrier properties to vapor permeation, it is preferable that the packed product scarcely suffers from any change in weight when stored at about 40° C./80% relative humidity (RH). The package material for the patch of the present invention preferably results in a weight change of the product of not more than about ±5%, when stored under the above-mentioned conditions for about 6 months.

Preparation

The mucoadhesive film (510) of the present invention can be prepared by numerous methods known in the art. In one embodiment, the components of the separate layers are separately dissolved in the appropriate solvent or combination of solvents to prepare a solution or suspension suitable for coating. Solvents for use in the present invention include, e.g., water, methanol, ethanol, or low alkyl alcohols such as isopropyl alcohol, acetone, methyl ethyl acetone, heptane, or dichloroethane, alone or in combination. The final solvent content or residual solvent content in the film can be the result of either or both layers.

The bioadhesive or backing solutions can then be separately coated onto an appropriate manufacturing substrate. Each solution is cast and processed into a thin film by techniques known in the art, such as by film dipping, film coating, film casting, spin coating, or spray drying using the appropriate substrate. The thin film is then dried. The drying step can be accomplished in any type of oven. However, the drying procedure should be selected to be compatible with the solvent employed and the amount of residual solvent may depend on the drying procedure. One of skill in the art can readily select appropriate drying procedures for the selected solvent(s). The film layers can be prepared independently and then laminated together or can be prepared as films, one sequentially coated on the top of the other.

The combined film obtained after the layers have been laminated together, or coated on top of each other, can be cut into any type of shape, for application to the tissue. The marginal outline of the ocular inserts can be triangular, oval circular, ring annular, reniform, square, ellipsoid, bean-shaped, rectangular, or any other symmetrical or unsymmetrical shape.

If the pharmaceutical(s) are added to the preformed mucoadhesive film (510) in a liquid form, i.e. postloaded, the solvent used to dissolve or suspend the pharmaceutical(s) can vary and typically depends upon the pharmaceutical(s) employed, as well as the other components of the mucoadhesive film (510). Typically, one of skill in the art can select a suitable solvent for the pharmaceutical(s) to be incorporated into the mucoadhesive film (510). Preferred solvents for the composition include organic-based solvents that have a high vapor pressure or a low normal boiling point and that have regulatory acceptance as a pharmaceutical solvent suitable for ocular administration. Examples of solvents that may be used include ethanol or isopropanol.

To postload a mucoadhesive film (510), an aliquot of the composition solution that includes a therapeutically effective amount of the pharmaceutical(s) is applied directly onto the chosen layer of the pre-assembled mucoadhesive film (510). Preferably, the layer is the bioadhesive layer. Dispensing equipment can be used for applying the pharmaceutical composition solution to the selected layer. Examples of microdispensing applicators that can be used include the IVEK® Precision Liquid Metering System. However, any suitable dispensing equipment can be employed. Examples of such dispensing equipment include precision syringes, pipetting equipment, and electronic fluid dispensers.

The aliquot is dried or otherwise stably adsorbed onto the surface of the selected layer to form a pharmaceutical-containing deposit on the surface of the mucoadhesive film (510). Drying of the dispensed solution is by any convenient means known to be acceptable for film drying. Examples of convenient drying methods include drying at ambient conditions or in a conventional film-drying oven. Alternatively, it may be desired for specific product characteristics to maintain the aliquot as a deposit liquid.

The postloaded composition can also be deposited in a solid form. Different solid forms can be used including films, powders, granules or tablets. The solid form can be prepared by forming a film that contains the pharmaceutical(s) and excipients. The film includes water-soluble polymers known to those of skill in the art, for example, some of the water-soluble polymers described herein. Each film can be prepared as a discrete unit, or the film can be divided into discrete units from a larger film, so that the individual films contain an efficacious amount of the pharmaceutical(s). Alternatively, the solid form of the composition can be prepared by compression of a powder mixture using procedures like those used to prepare pharmaceutical tablets. Other solid forms of the composition are suitable for application to the mucoadhesive film (510) of the present invention.

Desirable Uses of the Drug Delivery System

Systems made by the methods of the invention offer the advantages of an effective residence time with minimal discomfort and ease of use, and are an appropriate vehicle for the local as well as systemic delivery of pharmaceutical(s), given its thin, flexible form.

Systems formed by the methods of the invention are made of water-soluble components and are bioerodible. The use of water-soluble components allows the mucoadhesive film (510) to dissolve over a period of time, with natural bodily fluids slowly dissolving and eroding away the carrier, while the pharmaceutical(s) remain at the application site. Unlike bandages, transdermal devices and other non-water-soluble film systems, the user of the present invention does not have to remove the mucoadhesive film (510) following treatment. The user experiences minimal sensation of the presence of a foreign object at the conjunctival (330) surface of the eye, given that upon application, water absorption softens the mucoadhesive film (510), and over time, the mucoadhesive film (510) slowly dissolves or erodes away.

The residence times of water-soluble, bioerodible pharmaceutical mucoadhesive film (510)s made by the methods of the invention depend on the dissolution rate of the water-soluble polymers used. Dissolution rates may be adjusted by mixing together chemically different polymers, such as hydroxyethyl cellulose and hydroxypropyl cellulose; by using different molecular weight grades of the same polymer, such as mixing low and medium molecular weight hydroxyethyl cellulose; by using crosslinking agents such as glyoxal with polymers such as hydroxyethyl cellulose for partial crosslinking; by incorporating hydrophobic agents, such as mineral oil, into the backer formulation; or by post-treatment irradiation or curing, that may alter the physical state of the film, including its crystallinity or phase transition, once obtained. These strategies might be employed alone or in combination in order to modify the dissolution kinetics of the mucoadhesive film (510), without suppressing the water solubility characteristics of the component materials.

Upon application, the pharmaceutical delivery system adheres to the conjunctival (330) surface and remains in place. Water absorption softens the mucoadhesive film (510) quickly, diminishing and eliminating the foreign body sensation. As the system rests upon the conjunctival (330) or corneal surface, delivery of the pharmaceutical(s) is provided. Residence times may vary, depending on the formulation and materials used, but may be modulated between a few minutes to several hours.

The present invention includes the specific embodiments provided below:

[1] One embodiment of the present invention provides a mucoadhesive film (510) that includes:
a water-soluble bioadhesive layer (530) to be placed in contact with an ocular surface (560), the bioadhesive layer including one or more bioadhesive polymers and/or one or more film-forming, water-soluble polymers;
a water-soluble non-adhesive backing layer (540) that includes one or more water-soluble, film-forming, pharmaceutically acceptable polymers; and
one or more pharmaceuticals associated with the bioadhesive layer, associated with the non-adhesive layer, or associated with both the bioadhesive and non-adhesive layers;
wherein the mucoadhesive film (510) is compatible with ocular surfaces (560); the mucoadhesive film (510) adheres to ocular surfaces (560); the mucoadhesive film (510) is flexible; and the mucoadhesive film (510) is water-soluble, biodegradable, and bioerodible in tear fluids.

[2] Another embodiment of the present invention provides the mucoadhesive film (510) of embodiment [1] wherein the one or more film-forming water-soluble polymers includes an alkyl cellulose or a hydroxyalkyl cellulose.

[3] Another embodiment of the present invention provides the mucoadhesive film (510) of embodiment [1] wherein the one or more film-forming water-soluble polymers includes hydroxyethyl cellulose (HEC), hydroxypropyl cellulose (HPC), hydroxypropylmethyl cellulose (HPMC), hydroxyethylmethyl cellulose (HEMC), or a combination thereof.

[4] Another embodiment of the present invention provides the mucoadhesive film (510) of embodiment [1] wherein the one or more film-forming, water-soluble polymers includes hydroxypropylmethyl cellulose (HPMC).

[5] Another embodiment of the present invention provides the mucoadhesive film (510) of embodiment [4] wherein the hydroxypropylmethyl cellulose (HPMC) has an average molecular weight (Mw estimated from intrinsic viscosity measurements) in the range about 10² to about 10⁶.

[6] Another embodiment of the present invention provides the mucoadhesive film (510) of any one of embodiments [1]-[5] wherein the one or more film-forming water-soluble polymers are cross-linked.

[7] Another embodiment of the present invention provides the mucoadhesive film (510) of any one of embodiments [1]-[6] wherein the one or more film-forming water-soluble polymers are plasticized.

[8] Another embodiment of the present invention provides the mucoadhesive film (510) of any one of embodiments [1]-[6] wherein the water-soluble bioadhesive layer (530) is free of a plasticizer.

[9] Another embodiment of the present invention provides the mucoadhesive film (510) of any one of embodiments [1]-[8] wherein the one or more bioadhesive polymers include polyacrylic acid (PAA), sodium carboxymethyl cellulose (NaCMC), polyvinyl pyrrolidone (PVP), or a combination thereof.

[10] Another embodiment of the present invention provides the mucoadhesive film (510) of any one of embodiments [1]-[9] wherein the one or more water-soluble, film-forming, pharmaceutically acceptable polymers include an alkyl cellulose or a hydroxyalkyl cellulose.

[11] Another embodiment of the present invention provides the mucoadhesive film (510) of any one of embodiments [1]-[9] wherein the one or more water-soluble, film-forming, pharmaceutically acceptable polymers include hydroxyethyl cellulose (HEC), hydroxypropyl cellulose (HPC), hydroxypropylmethyl cellulose (HPMC), hydroxyethylmethyl cellulose (HEMC), polyvinylalcohol (PVA), polyethylene glycol (PEG), polyethylene oxide (PEO), ethylene oxide-propylene oxide co-polymers, or a combination thereof.

[12] Another embodiment of the present invention provides the mucoadhesive film (510) of any one of embodiments [1]-[9] wherein the one or more water-soluble, film-forming, pharmaceutically acceptable polymers include hydroxyethyl cellulose (HEC), hydroxypropyl cellulose (HPC), or a combination thereof.

[13] Another embodiment of the present invention provides the mucoadhesive film (510) of any one of embodiments [1]-[9] wherein the one or more water-soluble, film-forming, pharmaceutically acceptable polymers include hydroxyethyl cellulose (HEC).

[14] Another embodiment of the present invention provides the mucoadhesive film (510) of embodiment [13] wherein the hydroxyethyl cellulose (HEC) has an average molecular weight (Mw estimated from intrinsic viscosity measurements) in the range about 10² to about 10⁶.

[15] Another embodiment of the present invention provides the mucoadhesive film (510) of any one of embodiments [1]-[14] wherein the water-soluble non-adhesive backing layer (540) further includes a non-water soluble lubrication layer (520).

[16] Another embodiment of the present invention provides the mucoadhesive film (510) of embodiment [15] wherein the non-water soluble lubrication layer (520) includes an organosilicon-containing compound, a hydrocarbon, or a combination thereof.

[17] Another embodiment of the present invention provides the mucoadhesive film (510) of any one of embodiments [1]-[16] wherein the one or more pharmaceuticals are independently selected from the group of adrenergic agent; adrenocortical steroid; adrenocortical suppressant; alcohol deterrent; aldosterone antagonist; amino acid; ammonia detoxicant; anabolic; analeptic; analgesic; androgen; anesthesia, adjunt to; anesthetic; anorectic; antagonist; anterior pituitary suppressant; anthelmintic; antiacne agent; anti-adrenergic; anti-allergic; anti-amebic; anti-androgen; anti-anemic antianginal; anti-anxiety; anti-arthritic; anti-asthmatic; anti-atherosclerotic; antibacterial; anticholelithic; anticholelithogenic; anticholinergic; anticoagulant; anticoccidal; anticonvulsant; antidepressant; antidiabetic; antidiarrheal; antidiurietic; antidote; anti-emetic; anti-epileptic; anti-estrogen; antifibronolytic; antifungal; antiglaucoma agent; antihemophilic; antihermorrhagic; antihistamine; antihyperlipidemia; antihyperlipoproteinemic; antihypertensive; antihypotensive; anti-infctive; anti-infective, topical; anti-inflammatory; antikeratinizing agent; antimalarial; antimicrobial; antimigraine; antimycotic, antinausant, antineoplastic, antineutropenic, antiobessional agent; antiparasitic; antiparkinsonian; antiperistaltic, antipneumocystic; antiproliferative; antiprostatic hypertrophy; antiprotozoal; antipruritic; antipsychotic; antirheumatic; antischistosomal; antiseborrheic; antisecretory; antispasmodic; antithrombotic; antitussive; anti-ulcerative; anti-urolithic; antiviral; appetite suppressant; benign prostatic hyperplasia therapy agent; blood glucose regulator; bone resorption inhibitor; bronchodilator; carbonic anhydrase inhibitor; cardiac depressant; cardioprotectant; cardiotonic; cardiovascular agent; choleretic; cholinergic; cholinergie diagnostic aid; diuretic; dopaminergic agent; ectoparasiticide; emetic; enxzyme inhibitor; estrogen; fibrinolytic; flourescent agent; free oxygen radical scavenger; gastrointestinal motility effector; glucocorticoid; gonad-stimulating principle; hair growth stimulant; hemostatic; histamine H2 receptor antagonist; hormone; hypocholesterolemic; hypoglycemic; hypolipidemic; hypotensive; imaging agent; immunizing agent; immunomodulator; immunoregulator; immunostimulant; immunosuppressant; impotence therapy; inhibitor; keratolytic; LNRN agonist; liver disorder treatment; luteolysin; memory adjuvant; mental performance enhancer; mood regulator; mucolytic; mucosal protective agent; mydriatic; nasal decongestant; neuromuscular blocking agent; neuroprotective; NMDA antagonist; non-hormonal sterol derivative; oxytocic; plasminogen activator; platelet activating factor antagonist; platelet aggregaton inhibitor; post-stroke and post-head trauma treatment; potentiator; progestin; prostaglandin; prostate growth inhibitor; prothyrotropin; psychotropic; radioactive agent; regulator; relaxant; repartitioning agent; scabicide; sclerosing agent; sedative; sedative-hypnotic; selective adenosine A1 antagonist; serotonin antagonist; serotinin inhibitor; serotinin receptor antagonist; steroid; stimulant; suppressant; symptomatic multiple sclerosis; synergist; thyroid hormone; thyroid inhibitor; thyromimetic; tranquilizer; treatment of amyotrophic laterial sclerosis; treatment of cerebral ischemia; treatment of Paget's disease; treatment of unstable angina; uricosuric; vasoconstrictor; vasodilator; vulnerary; wound healing agent; zxanthine oxidase inhibitor; and combinations thereof.

[18] Another embodiment of the present invention provides the mucoadhesive film (510) of any one of embodiments [1]-[16] wherein the one or more pharmaceuticals are selected from the group of Acebutolol; Acebutolol; Acyclovir; Albuterol; Alfentanil; Almotriptan; Alprazlam; Amiodarone; Amlexanox; Amphotericin B; Atorvastatin; Atropine; Auranofin; Aurothioglucose; Benazepril; Bicalutamide; Bretylium; Brifentanil; Bromocriptine; Buprenorphine; Butorphanol; Buspirone; Calcitonin; Candesartan; Carfentanil; Carvedilol; Chlorpheniramine; Chlorothiazide; Chlorphentermine; Chlorpromazine; Clindamycin; Clonidine; Codeine; Cyclosporine; Desipramine; Desmopressin; Dexamethasone; Diazepam; Diclofenac; Digoxin; Digydrocodeine; Dolasetron; Dopamine; Doxepin; Doxycycline; Dronabinol; Droperidol; Dyclonine; Eletriptan; Enalapril; Enoxaparin; Ephedrine; Epinephrine; Ergotamine; Etomidate; Famotidine; Felodipine; Fentanyl; Fexofenadine; Fluconazole; Fluoxetine; Fluphenazine; Flurbiprofen; Fluvastatin; Fluvoxamine; Frovatriptan; Furosemide; Ganciclovir; Gold sodium thiomalate; Granisetron; Griseofulvin; Haloperidol; Hepatitis B Virus Vaccine; Hydralazine; Hydromorphone; Insulin; Ipratropium; Isradipine; Isosorbide Dinitrate; Ketamine; Ketorolac; Labetalol; Levorphanol; Lisinopril; Loratadine; Lorazepam; Losartan; Lovastatin; Melatonin; Methyldopa; Methylphenidate; Metoprolol; Midazolam; Mirtazapine; Morhpine; Nadolol; Nalbuphine; Naloxone; Naltrexone; Naratriptan; Neostgmine; Nicardipine; Nifedipine; Norepinephrine; Nortriptyline; Octreotide; Olanzapine; Omeprazole; Ondansetron; Oxybutynin; Oxycodone; Oxymorphone; Oxytocin; Phenylephrine; Phenylpropanolaimine; Phenytoin; Pimozide; Pioglitazone; Piroxicam; Pravastatin; Prazosin; Prochlorperazine; Propafenone; Prochlorperazine; Propiomazine; Propofol; Propranolol; Pseudoephedrine; Pyridostigmine; Quetiapine; Raloxifene; Remifentanil; Rofecoxib; repaglinide; Risperidone; Rizatriptan; Ropinirole; Scopolamine; Selegiline; Sertraline; Sildenafil; Simvastatin; Sirolimus; Spironolactone; Sufentanil; Sumatriptan; Tacrolimus; Tamoxifen; Terbinafine; Terbutaline; Testosterone; Tetanus toxoid; THC Tolterodine; Triamterene; Triazolam; Tricetamide; Valsartan; Venlafaxine; Verapamil; Zaleplon; Zanamivir; Zafirlukast; Zolmitriptan; Zolpidem; and combinations thereof.

[19] Another embodiment of the present invention provides the mucoadhesive film (510) of any one of embodiments [1]-[16] wherein the one or more pharmaceuticals are selected from the group of naratriptan, zolmitriptan, rizatriptan, frovatriptan, sumatriptan, and combinations thereof.

[20] Another embodiment of the present invention provides the mucoadhesive film (510) of any one of embodiments [1]-[16] wherein the one or more pharmaceuticals are an antiangiogenic agent to the retinochoroid.

[21] Another embodiment of the present invention provides the mucoadhesive film (510) of any one of embodiments [1]-[16] wherein the one or more pharmaceuticals are an immunosuppressive agent.

[22] Another embodiment of the present invention provides the mucoadhesive film (510) of any one of embodiments [1]-[16] wherein the one or more pharmaceuticals are an anti-inflammatory agent.

[23] Another embodiment of the present invention provides the mucoadhesive film (510) of any one of embodiments [1]-[16] wherein the one or more pharmaceuticals are an antibacterial agent.

[24] Another embodiment of the present invention provides the mucoadhesive film (510) of any one of embodiments [1]-[16] wherein the one or more pharmaceuticals are an antiviral agent.

[25] Another embodiment of the present invention provides the mucoadhesive film (510) of any one of embodiments [1]-[16] wherein the one or more pharmaceuticals are an antifungal agent.

[26] Another embodiment of the present invention provides the mucoadhesive film (510) of any one of embodiments [1]-[16] wherein the one or more pharmaceuticals are present in a combined amount of up to about 30 wt. % of the mucoadhesive film (510).

[27] Another embodiment of the present invention provides the mucoadhesive film (510) of any one of embodiments [1]-[16] wherein the one or more pharmaceuticals are present in a combined amount of up between about 0.005 wt. % and about 20 wt. % of the mucoadhesive film (510).

[28] Another embodiment of the present invention provides the mucoadhesive film (510) of any one of embodiments [1]-[16] wherein the one or more pharmaceuticals are independently located uniformly throughout the bioadhesive layer, uniformly throughout the non-adhesive layer, or uniformly throughout both the bioadhesive and the non-adhesive layers.

[29] Another embodiment of the present invention provides the mucoadhesive film (510) of any one of embodiments [1]-[16] wherein the one or more pharmaceuticals are independently located uniformly throughout the bioadhesive layer.

[30] Another embodiment of the present invention provides the mucoadhesive film (510) of any one of embodiments [1]-[16] wherein the one or more pharmaceuticals are independently located near the center of the bioadhesive layer.

[31] Another embodiment of the present invention provides mucoadhesive film (510) of any one of embodiments [1]-[30] wherein the one or more pharmaceuticals are locally delivered to the ocular region (550).

[32] Another embodiment of the present invention provides mucoadhesive film (510) of any one of embodiments [1]-[30] wherein the one or more pharmaceuticals are systemically delivered via the ocular surface (560).

[33] Another embodiment of the present invention provides the mucoadhesive film (510) of any one of embodiments [1]-[32] having a thickness of up to about 1 mm.

[34] Another embodiment of the present invention provides the mucoadhesive film (510) of any one of embodiments [1]-[32] having a thickness of between about 0.1 mm to about 0.5 mm.

[35] Another embodiment of the present invention provides the mucoadhesive film (510) of any one of embodiments [1]-[34] further including a pharmaceutically acceptable dissolution-rate-modifying agent, pharmaceutically acceptable disintegration aid, pharmaceutically acceptable plasticizer, pharmaceutically acceptable coloring agent, pharmaceutically acceptable opaquifier, pharmaceutically acceptable anti-oxidant, pharmaceutically acceptable film forming enhancer, pharmaceutically acceptable preservative, or a combination thereof.

[36] Another embodiment of the present invention provides the mucoadhesive film (510) of any one of embodiments [1]-[35] wherein the ocular surface (560) is the conjunctival tissue.

[37] Another embodiment of the present invention provides the mucoadhesive film (510) of embodiment [36] wherein the conjunctival tissue is the bulbar conjunctiva (220), the palpebral conjunctiva (230), the inferior palperbral conjunctiva, or a combination thereof.

[38] Another embodiment of the present invention provides the mucoadhesive film (510) of any one of embodiments [1]-[35] wherein the ocular surface (560) is the corneal tissue.

[39] Another embodiment of the present invention provides the mucoadhesive film (510) of embodiment [38] wherein the corneal tissue is the corneal epithelium.

[40] Another embodiment of the present invention provides the mucoadhesive film (510) of any one of embodiments [1]-[35] wherein the ocular surface (560) is surface epithelial tissue of the eye.

[41] Another embodiment of the present invention provides the mucoadhesive film (510) of any one of embodiments [1]-[35] wherein the ocular surface (560) is the inside of the eyelid.

[42] Another embodiment of the present invention provides the mucoadhesive film (510) of any one of embodiments [1]-[41] further including a third layer located between the water-soluble bioadhesive layer (530) and the water-soluble non-adhesive backing layer (540); wherein the third layer is flexible, biodegradable, bioerodible in tear fluids, and water-soluble.

[43] Another embodiment of the present invention provides the mucoadhesive film (510) of any one of embodiments [1]-[42] further including a component that acts to adjust the kinetics of the erodability of the mucoadhesive film (510).

[44] Another embodiment of the present invention provides the mucoadhesive film (510) of embodiment [43] wherein the component is a water-based emulsion of polylactide, polyglycolide, lactide-glycolide copolymers, poly-e-caprolactone, polyorthoesters, polyanhydrides, ethyl cellulose, vinyl acetate, cellulose, acetate, polyisobutylene, or combinations thereof.

[45] Another embodiment of the present invention provides a method for treating a wound on an ocular surface (560) of a mammal including contacting the ocular surface (560) of the mammal afflicted with the wound, a mucoadhesive film (510) of any one of embodiments [1]-[44].

[46] Another embodiment of the present invention provides a method for delivering a pharmaceutical to an ocular surface (560) of a mammal including contacting the ocular surface (560) of the mammal with a mucoadhesive film (510) of any one of embodiments [1]-[44].

[47] Another embodiment of the present invention provides the method of embodiment [46] wherein the delivery of the pharmaceutical is local.

[48] Another embodiment of the present invention provides the method of embodiment [46] wherein the delivery of the pharmaceutical is systemic.

[49] Another embodiment of the present invention provides a method for treating a mammal suffering from a migraine headache, the method including contacting an ocular surface (560) of the mammal afflicted with the migraine headache, a mucoadhesive film (510) of any one of embodiments [1]-[44].

[50] Another embodiment of the present invention provides a method for treating a mammal suffering from diabetic retinopathy, the method including contacting an ocular surface (560) of the mammal afflicted with the diabetic retinopathy, a mucoadhesive film (510) of any one of embodiments [1]-[44].

[51] Another embodiment of the present invention provides a method for treating a mammal suffering from muscular degeneration, the method including contacting an ocular surface (560) of the mammal afflicted with the muscular degeneration, a mucoadhesive film (510) of any one of embodiments [1]-[44].

[52] Another embodiment of the present invention provides a method for treating a mammal suffering from uveitis, the method including contacting an ocular surface (560) of the mammal afflicted with the uveitis, a mucoadhesive film (510) of any one of embodiments [1]-[44].

[53] Another embodiment of the present invention provides a method for treating a mammal suffering from herpetic conjunctivitis, the method including contacting an ocular surface (560) of the mammal afflicted with the herpetic conjunctivitis, a mucoadhesive film (510) of any one of embodiments [1]-[44].

[54] Another embodiment of the present invention provides a method for treating a mammal suffering from blepharitis, the method including contacting an ocular surface (560) of the mammal afflicted with the blepharitis, a mucoadhesive film (510) of any one of embodiments [1]-[44].

[55] Another embodiment of the present invention provides the method of any one of embodiments [45]-[54] wherein the residence time is up to about 7 days.

[56] Another embodiment of the present invention provides the method of any one of embodiments [45]-[54] wherein the residence time is up to about 24 hours.

[57] Another embodiment of the present invention provides the method of any one of embodiments [45]-[54] wherein the residence time is up to about 8 hours.

[58] Another embodiment of the present invention provides the method of any one of embodiments [45]-[54] wherein the residence time is between about 1 minute and about 4 hours.

[59] Another embodiment of the present invention provides a method for locally delivering one or more pharmaceuticals to an ocular region (550) of a mammal, the method including contacting the ocular surface (560) of the mammal with a mucoadhesive film (510) of any one of embodiments 1-31 and 33-44.

[60] Another embodiment of the present invention provides a method for systemically delivering one or more pharmaceuticals to a mammal via an ocular surface (560), the method including contacting the ocular surface (560) of the mammal with a mucoadhesive film (510) of any one of embodiments 1-30 and 32-44.

The examples are intended to further illustrate, but not limit, the invention. These examples illustrate compositions for the transconjunctival or transcorneal delivery of pharmaceuticals for either local or systemic therapy. The following examples also illustrate the ability of the conjunctiva to provide rapid onset of therapeutic action and increased bioavailability compared to eye drops.

Those skilled in the art will recognize that, while specific embodiments have been illustrated and described, various modifications and changes may be made without departing from the spirit and scope of the invention.

### EXAMPLE 1

A 200 gm batch of BEMA™ backing stock was manufactured on a weight per weight basis of: 77% purified water, 11% hydroxyethyl cellulose, 11% hydroxypropyl cellulose, and 1% tocopheryl acetate. All materials were mixed until the batch was homogeneous.

A 200 gram batch of water-soluble bioadhesive was made by mixing on a weight per weight basis: 90.0% purified water, 5.5% hydroxypropylmethyl cellulose, 4.4% hydroxyethyl cellulose, and 0.1% tocopheryl acetate. Mixing was performed until all components were homogeneous.

### EXAMPLE 2.

Using the stock solutions of example 1, a Frovatriptan bioerodible adhesive drug delivery system was fabricated. A 6.5% weight per weight basis of frovatriptan succinate was compounded in the adhesive stock by mixing 9.39 grams of bioadhesive and 0.65 grams of frovatriptan succinate. The stock was mixed in a Flak Tek mixer for 5 minutes at 3000 rpm, which produced a homogenous solution.

Using a Werner Mathis Labcoater, the substrate, siliconized Mylar, (Rexam 3 mil PET 92A/000), was secured, and the backing layer solution was set in front of a knife over-roll with an opening (wet gap) of 0.10 mm. The backing solution was coated and the film dried for 3.5 minutes at 90° C. The drug loaded bioadhesive was coated over the dried backer film with a wet gap of 0.50 mm and dried for 5 minutes at 90° C. The BEMA™ film was cut with a rounded square die cutter (10 mm×10 mm).

A single rounded square frovatriptan delivery system was placed in the right eye of a dog with the adhesive side of the bioerodible film adhered to the inferior palpebral conjunctiva (230). This was repeated in five separate dogs with plasma levels of frovatriptan being determined five minutes after application. Four of the five dogs had plasma levels of 4 nanograms per milliliter or higher five minutes after administration of the delivery system. Plasma concentrations of 4 nanograms per milliliter or higher in the dog are considered to represent therapeutic plasma levels of frovatriptan.

### EXAMPLE 3.

Using the stock solutions of example 1, a sumatriptan bioerodible adhesive drug delivery system was fabricated. A 12% weight per weight basis of sumatriptan succinate was compounded in the adhesive stock by mixing 17.6 grams of bioadhesive and 2.4 grams sumatriptan succinate. The stock was mixed in Flak Tek mixer for 5 minutes at 3000 rpm, which produced a homogenous solution.

Using a Werner Mathis Labcoater, the substrate, siliconized Mylar, (Rexam 3 mil PET 92A/000), was secured, and the backing layer solution was set in front of a knife over-roll with an opening (wet gap) of 0.10 mm. The backing solution was then coated and the film dried for 3.5 minutes at 90° C. The bioadhesive with drug was coated over the dried backer film with a wet gap of 0.50 mm and dried for 5 minutes at 90° C. The BEMA™ film was cut with a rounded square die cutter (10 mm×10 mm).

### EXAMPLE 4.

Using the stock solutions of example 1, a naratriptan bioerodible adhesive drug delivery system was fabricated. A 7% weight per weight basis of naratriptan hydrochloride was compounded in the adhesive stock by mixing 18.6 grams of bioadhesive and 1.4 grams of Naratriptan HCl. The stock was mixed in a Flak Tek mixer for 5 minutes at 3000 rpm, which produced a homogenous solution.

Using a Werner Mathis Labcoater, the substrate, siliconized Mylar, (Rexam 3 mil PET 92A/000), was secured, and the backing layer solution was set in front of a knife over-roll with an opening (wet gap) of 0.10 mm. The backing solution was coated and the film dried for 3.5 minutes at 90° C. The drug loaded bioadhesive was coated over the dried backer film with a wet gap of 0.50 mm and dried for 5 minutes at 90° C. The BEMA™ film was cut with a rounded square die cutter (10 mm×10 mm).

All patents, patent documents, and references cited herein form part of the invention.

1. A method for delivering a pharmaceutical via an ocular surface of a mammal, the method comprising contacting the ocular surface of the mammal with a mucoadhesive film that comprises:
a water-soluble bioadhesive layer to be placed in contact with an ocular surface, the bioadhesive layer including one or more bioadhesive polymers and/or one or more film-forming, water-soluble polymers;
a water-soluble non-adhesive backing layer that comprises one or more water-soluble, film-forming, pharmaceutically acceptable polymers; and
one or more pharmaceuticals associated with the bioadhesive layer, associated with the non-adhesive layer, or associated with both the bioadhesive and non-adhesive layers;
wherein the mucoadhesive film is compatible with ocular surfaces; the mucoadhesive film adheres to ocular surfaces; the mucoadhesive film is flexible; and the mucoadhesive film is water-soluble, biodegradable, and bioerodible in tear fluids.

2. The method of claim 1 wherein the one or more film-forming water-soluble polymers comprises an alkyl cellulose or a hydroxyalkyl cellulose.

3. The method of claim 1 wherein the one or more film-forming water-soluble polymers comprises hydroxyethyl cellulose (HEC), hydroxypropyl cellulose (HPC), hydroxypropylmethyl cellulose (HPMC), hydroxyethylmethyl cellulose (HEMC), or a combination thereof.

4. The method of claim 1 wherein the one or more film-forming, water-soluble polymers comprises hydroxypropylmethyl cellulose (HPMC).

5. The method of claim 1 wherein the one or more bioadhesive polymers comprise polyacrylic acid (PAA), sodium carboxymethyl cellulose (NaCMC), polyvinyl pyrrolidone (PVP), or a combination thereof.

6. The method of claim 1 wherein the one or more water-soluble, film-forming, pharmaceutically acceptable polymers comprise an alkyl cellulose or a hydroxyalkyl cellulose.

7. The method of claim 1 wherein the one or more water-soluble, film-forming, pharmaceutically acceptable polymers comprise hydroxyethyl cellulose (HEC), hydroxypropyl cellulose (HPC), hydroxypropylmethyl cellulose (HPMC), hydroxyethylmethyl cellulose (HEMC), polyvinylalcohol (PVA), polyethylene glycol (PEG), polyethylene oxide (PEO), ethylene oxide-propylene oxide co-polymers, or a combination thereof.

8. The method of claim 1 wherein the one or more water-soluble, film-forming, pharmaceutically acceptable polymers comprise hydroxyethyl cellulose (HEC), hydroxypropyl cellulose (HPC), or a combination thereof.

9. The method of claim 1 wherein the one or more water-soluble, film-forming, pharmaceutically acceptable polymers comprise hydroxyethyl cellulose (HEC).

10. The method of claim 1 wherein the water-soluble non-adhesive backing layer further comprises a non-water soluble lubrication layer.

11. The method of claim 1 wherein the one or more pharmaceuticals are independently selected from the group of adrenergic agent; adrenocortical steroid; adrenocortical suppressant; alcohol deterrent; aldosterone antagonist; amino acid; ammonia detoxicant; anabolic; analeptic; analgesic; androgen; anesthesia, adjunt to; anesthetic; anorectic; antagonist; anterior pituitary suppressant; anthelmintic; antiacne agent; anti-adrenergic; anti-allergic; anti-amebic; anti-androgen; anti-anemic antianginal; anti-anxiety; anti-arthritic; anti-asthmatic; anti-atherosclerotic; antibacterial; anticholelithic; anticholelithogenic; anticholinergic; anticoagulant; anticoccidal; anticonvulsant; antidepressant; antidiabetic; antidiarrheal; antidiurietic; antidote; anti-emetic; anti-epileptic; anti-estrogen; antifibronolytic; antifungal; antiglaucoma agent; antihemophilic; antihermorrhagic; antihistamine; antihyperlipidemia; antihyperlipoproteinemic; antihypertensive; antihypotensive; anti-infctive; anti-infective, topical; anti-inflammatory; antikeratinizing agent; antimalarial; antimicrobial; antimigraine; antimycotic, antinausant, antineoplastic, antineutropenic, antiobessional agent; antiparasitic; antiparkinsonian; antiperistaltic, antipneumocystic; antiproliferative; antiprostatic hypertrophy; antiprotozoal; antipruritic; antipsychotic; antirheumatic; antischistosomal; antiseborrheic; antisecretory; antispasmodic; antithrombotic; antitussive; anti-ulcerative; anti-urolithic; antiviral; appetite suppressant; benign prostatic hyperplasia therapy agent; blood glucose regulator; bone resorption inhibitor; bronchodilator; carbonic anhydrase inhibitor; cardiac depressant; cardioprotectant; cardiotonic; cardiovascular agent; choleretic; cholinergic; cholinergie diagnostic aid; diuretic; dopaminergic agent; ectoparasiticide; emetic; enxzyme inhibitor; estrogen; fibrinolytic; flourescent agent; free oxygen radical scavenger; gastrointestinal motility effector; glucocorticoid; gonad-stimulating principle; hair growth stimulant; hemostatic; histamine H2 receptor antagonist; hormone; hypocholesterolemic; hypoglycemic; hypolipidemic; hypotensive; imaging agent; immunizing agent; immunomodulator; immunoregulator; immunostimulant; immunosuppressant; impotence therapy; inhibitor; keratolytic; LNRN agonist; liver disorder treatment; luteolysin; memory adjuvant; mental performance enhancer; mood regulator; mucolytic; mucosal protective agent; mydriatic; nasal decongestant; neuromuscular blocking agent; neuroprotective; NMDA antagonist; non-hormonal sterol derivative; oxytocic; plasminogen activator; platelet activating factor antagonist; platelet aggregaton inhibitor; post-stroke and post-head trauma treatment; potentiator; progestin; prostaglandin; prostate growth inhibitor; prothyrotropin; psychotropic; radioactive agent; regulator; relaxant; repartitioning agent; scabicide; sclerosing agent; sedative; sedative-hypnotic; selective adenosine A1 antagonist; serotonin antagonist; serotinin inhibitor; serotinin receptor antagonist; steroid; stimulant; suppressant; symptomatic multiple sclerosis; synergist; thyroid hormone; thyroid inhibitor; thyromimetic; tranquilizer; treatment of amyotrophic laterial sclerosis; treatment of cerebral ischemia; treatment of Paget's disease; treatment of unstable angina; uricosuric; vasoconstrictor; vasodilator; vulnerary; wound healing agent; zxanthine oxidase inhibitor; and combinations thereof.

12. The method of claim 1 wherein the one or more pharmaceuticals are selected from the group of Acebutolol; Acebutolol; Acyclovir; Albuterol; Alfentanil; Almotriptan; Alprazlam; Amiodarone; Amlexanox; Amphotericin B; Atorvastatin; Atropine; Auranofin; Aurothioglucose; Benazepril; Bicalutamide; Bretylium; Brifentanil; Bromocriptine; Buprenorphine; Butorphanol; Buspirone; Calcitonin; Candesartan; Carfentanil; Carvedilol; Chlorpheniramine; Chlorothiazide; Chlorphentermine; Chlorpromazine; Clindamycin; Clonidine; Codeine; Cyclosporine; Desipramine; Desmopressin; Dexamethasone; Diazepam; Diclofenac; Digoxin; Digydrocodeine; Dolasetron; Dopamine; Doxepin; Doxycycline; Dronabinol; Droperidol; Dyclonine; Eletriptan; Enalapril; Enoxaparin; Ephedrine; Epinephrine; Ergotamine; Etomidate; Famotidine; Felodipine; Fentanyl; Fexofenadine; Fluconazole; Fluoxetine; Fluphenazine; Flurbiprofen; Fluvastatin; Fluvoxamine; Frovatriptan; Furosemide; Ganciclovir; Gold sodium thiomalate; Granisetron; Griseofulvin; Haloperidol; Hepatitis B Virus Vaccine; Hydralazine; Hydromorphone; Insulin; Ipratropium; Isradipine; Isosorbide Dinitrate; Ketamine; Ketorolac; Labetalol; Levorphanol; Lisinopril; Loratadine; Lorazepam; Losartan; Lovastatin; Melatonin; Methyldopa; Methylphenidate; Metoprolol; Midazolam; Mirtazapine; Morhpine; Nadolol; Nalbuphine; Naloxone; Naltrexone; Naratriptan; Neostgmine; Nicardipine; Nifedipine; Norepinephrine; Nortriptyline; Octreotide; Olanzapine; Omeprazole; Ondansetron; Oxybutynin; Oxycodone; Oxymorphone; Oxytocin; Phenylephrine; Phenylpropanolaimine; Phenytoin; Pimozide; Pioglitazone; Piroxicam; Pravastatin; Prazosin; Prochlorperazine; Propafenone; Prochlorperazine; Propiomazine; Propofol; Propranolol; Pseudoephedrine; Pyridostigmine; Quetiapine; Raloxifene; Remifentanil; Rofecoxib; repaglinide; Risperidone; Rizatriptan; Ropinirole; Scopolamine; Selegiline; Sertraline; Sildenafil; Simvastatin; Sirolimus; Spironolactone; Sufentanil; Sumatriptan; Tacrolimus; Tamoxifen; Terbinafine; Terbutaline; Testosterone; Tetanus toxoid; THC Tolterodine; Triamterene; Triazolam; Tricetamide; Valsartan; Venlafaxine; Verapamil; Zaleplon; Zanamivir; Zafirlukast; Zolmitriptan; Zolpidem; and combinations thereof.

13. The method of claim 1 wherein the one or more pharmaceuticals are present in a combined amount of up between about 0.005 wt. % and about 20 wt. % of the mucoadhesive film.

14. The method of claim 1 wherein the mucoadhesive film has a thickness of between about 0.1 mm to about 0.5 mm.

15. The method of claim 1 wherein the mucoadhesive film further includes a pharmaceutically acceptable dissolution-rate-modifying agent, a pharmaceutically acceptable disintegration aid, a pharmaceutically acceptable plasticizer, a pharmaceutically acceptable coloring agent, a pharmaceutically acceptable opaquifier, a pharmaceutically acceptable anti-oxidant, a pharmaceutically acceptable film forming enhancer, a pharmaceutically acceptable preservative, a component that acts to adjust the kinetics of the erodability of the mucoadhesive film, or a combination thereof.

16. The method of claim 1 wherein the mucoadhesive film further includes a third layer located between the water-soluble bioadhesive layer and the water-soluble non-adhesive backing layer; wherein the third layer is flexible, biodegradable, bioerodible in tear fluids, and water-soluble.

17. The method of claim 1 wherein the pharmaceutical is locally delivered.

18. The method of claim 1 wherein the pharmaceutical is systemically delivered.

19. A method for locally delivering a pharmaceutical via an ocular surface of a mammal, the method comprising contacting the ocular surface of the mammal with a mucoadhesive film that comprises:
a water-soluble bioadhesive layer to be placed in contact with an ocular surface, the bioadhesive layer including one or more bioadhesive polymers and/or one or more film-forming, water-soluble polymers;
a water-soluble non-adhesive backing layer that comprises one or more water-soluble, film-forming, pharmaceutically acceptable polymers; and
one or more pharmaceuticals associated with the bioadhesive layer, associated with the non-adhesive layer, or associated with both the bioadhesive and non-adhesive layers;
wherein the mucoadhesive film is compatible with ocular surfaces; the mucoadhesive film adheres to ocular surfaces; the mucoadhesive film is flexible; and the mucoadhesive film is water-soluble, biodegradable, and bioerodible in tear fluids.

20. A method for systemically delivering a pharmaceutical via an ocular surface of a mammal, the method comprising contacting the ocular surface of the mammal with a mucoadhesive film that comprises:
a water-soluble bioadhesive layer to be placed in contact with an ocular surface, the bioadhesive layer including one or more bioadhesive polymers and/or one or more film-forming, water-soluble polymers;
a water-soluble non-adhesive backing layer that comprises one or more water-soluble, film-forming, pharmaceutically acceptable polymers; and
one or more pharmaceuticals associated with the bioadhesive layer, associated with the non-adhesive layer, or associated with both the bioadhesive and non-adhesive layers;
wherein the mucoadhesive film is compatible with ocular surfaces; the mucoadhesive film adheres to ocular surfaces; the mucoadhesive film is flexible; and the mucoadhesive film is water-soluble, biodegradable, and bioerodible in tear fluids.
